(19) United States
(12) Patent Application Publication
(10) Pub. No.: US 2025/0171818 A1
(43) Pub. Date: May 29, 2025

(54) MIXED MICROBIAL AGENT WITH HIGH YIELD OF BACTERIAL CELLULOSE AND METHOD FOR PRODUCING NATA FIBER USING MIXED MICROBIAL AGENT

(71) Applicant: FUJIAN QUANZHOU XIDUODUO FOOD CO., LTD., Quanzhou (CN)

(72) Inventors: Wei FENG, Quanzhou (CN); Qingchun XU, Quanzhou (CN); Huijuan ZHAO, Quanzhou (CN); Yu QIN, Quanzhou (CN); Lina LIU, Quanzhou (CN); Donghui XU, Quanzhou (CN); Pingping ZENG, Quanzhou (CN)

(73) Assignee: FUJIAN QUANZHOU XIDUODUO FOOD CO., LTD., Quanzhou (CN)

(21) Appl. No.: 19/026,538

(22) Filed: Jan. 17, 2025

Related U.S. Application Data
(63) Continuation-in-part of application No. PCT/CN2023/108828, filed on Jul. 24, 2023.

(30) Foreign Application Priority Data
Jan. 29, 2023 (CN) 202310081664.4

(51) Int. Cl. C12P 19/04; C12N 1/20; C12R 1/01

(52) U.S. Cl. CPC C12P 19/04; C12N 1/205; C12R 2001/01

(57) ABSTRACT

A mixed microbial agent with a high yield of bacterial cellulose is provided. The mixed microbial agent includes Komagataeibacter oboediens with a deposit number of CCTCC NO: M 20221774 and Leuconostoc mesenteroides with a deposit number of CCTCC NO: M 20221775. The mass ratio of Komagataeibacter oboediens to Leuconostoc mesenteroides is 1:(0.1-0.5), and the total viable count is 10⁷-10⁸ cfu/mL. The mixed microbial agent is used in the process of producing Nata fiber, the growth environment and the fermentation method of the strain are optimized, and the yield and quality of the Nata fiber can be improved.

### CROSS REFERENCE TO THE RELATED APPLICATIONS

This application is a continuation-in-part application of International Application No. PCT/CN2023/108828, filed on Jul. 24, 2023, which is based upon and claims priority to Chinese Patent Application No. 202310081664.4, filed on Jan. 29, 2023, the entire contents of which are incorporated herein by reference.

### TECHNICAL FIELD

The present disclosure relates to the technical field of microbial fermentation, in particular to a mixed microbial agent with a high yield of bacterial cellulose and a method for producing Nata fiber using the mixed microbial agent.

### BACKGROUND

Bacterial cellulose (also known as Nata fiber in food field) is a natural cellulose produced by fermentation of certain specific microbial genera and species in a static or dynamic environment and is an extracellular product produced by bacteria. Bacterial cellulose is an unbranched, macromolecular, straight-chain polymer formed by connecting pyran glucose monomers through β-1,4-glycosidic bonds, which has the composition of (C₆H₁₀O₅)n, and the straight chains are parallel to each other, without helical conformation and branching structure. Acetobacter xylimm, as the strain with the strongest ability to produce bacterial cellulose, has been widely studied at home and abroad.

Bacterial cellulose is a gel-like, hydrophilic polysaccharide, is a homogeneous glucan linked by β-1,4-glycosidic bonds, and is an ideal new biodegradable polymer. Bacterial cellulose has many unique properties, including strong water-holding capacity, high purity, super fineness, high Young's modulus, super performance and shape plasticity, high hydrophilicity, high air and water permeability, good biocompatibility and biodegradability, and controllability of shape and performance during biosynthesis.

Because of its many unique properties, bacterial cellulose is currently recognized as a new biomaterial with excellent performance in the world. It has broad and high added value applications in medicine, chemical industry, food, and other fields, such as wound dressings, artificial blood vessels, tissue engineering scaffolds and other biomedical fields, papermaking, non-woven fabrics, petroleum exploitation and other chemical fields. It can be widely used as thickener, binder, molding agent, dispersant, and dietary fiber food in the food field.

At present, there are still some problems in the industrial production of bacterial cellulose, such as a single available carbon source, low yield, a long growth cycle, and poor fermentation environment. Some patents relate to improving the yield of bacterial cellulose. Patent CN106497789 discloses a screening method of bacterial strains with high bacterial cellulose production capacity; Patent CN103060229 discloses a method for efficiently producing bacterial cellulose by fermenting Acetobacter xylinum and mixed culture; Patent CN104372048 discloses a method for improving the yield of bacterial cellulose produced by Acetobacter xylimim fermentation. All the above patents have the problems that the screened strains are still not suitable for industrial production, and the raw material cost is high, the operation is complicated, and the production cycle is long. In a large number of patents and literature, the production of bacterial cellulose generally adopts shallow tray static fermentation, which is not confined and easy to be affected by other miscellaneous bacteria and is greatly affected by environmental factors.

### SUMMARY

### Technical Problems

Bacterial cellulose has a wide range of commercial application prospects but also has high production costs and great limitations at present. Increasing the production of bacterial cellulose is conducive to reducing the cost and expanding the application, and improving the fermentation stability is conducive to further improving the economic benefit.

### Technical Solutions

Aiming at the above problems existing in the prior art, the present disclosure provides a mixed microbial agent with a high yield of bacterial cellulose and a method for producing Nata fiber using the mixed microbial agent. The present disclosure can improve the yield and quality of Nata fiber by optimizing the growth environment of the strain and the fermentation method.

The technical solution of the present disclosure is as follows:

The first purpose of the present disclosure is to provide a mixed microbial agent with a high yield of bacterial cellulose, including Komagataeibacter oboediens and Leuconostoc mesenteroides, where the mass ratio of Komagataeibacter oboediens and Leuconostoc mesenteroides is 1:(0.1-0.5), and the total viable count is 10⁷-10⁸ cfu/mL.

In one embodiment of the present disclosure, the deposit number of Komagataeibacter oboediens JGG1 is CCTCC NO: M 20221774.

In one embodiment of the present disclosure, the Leuconostoc mesenteroides is named as Leuconostoc mesenteroides subsp. mesenteroides JGG2, and the deposit number is CCTCC NO: M 20221775.

The second purpose of the present disclosure is to provide a method for producing Nata fiber by using the mixed microbial agent, including the following steps:

(1) seed culture preparation: inoculating Komagataeibacter oboediens and Leuconostoc mesenteroides into test tubes containing expansion culture medium, respectively, increasing the yield by 10-20 times per generation, culturing at 25° C.-35° C. for 2-4 d per generation, and obtaining required seed culture through 2-5 generations of subculture, where the bacterial concentration of the seed culture is 10⁷-10⁸ cfu/mL;

(2) inoculation and fermentation: mixing the seed cultures of Komagataeibacter oboediens and Leuconostoc mesenteroides according to the mass ratio of 1:(0.1-0.5), preparing a fermentation medium, inoculating the seed cultures according to 1%-20% by weight, after stirring, filling the fermentation solution into a container box, and conducting static culture at 25° C.-35° C. for 6-8 g to obtain a high yield of Nata fiber.

In one embodiment of the present disclosure, the expansion culture medium required for the preparation of seed culture includes 10-100 g/L carbon source, 0.5-20 g/L nitrogen source, and 0.5-20 g/L inorganic salt, and the initial pH range is 2.5-6.5; and the culture method is shaking culture or static culture.

The carbon source and nitrogen source in the expansion culture medium are supplemented by 0.8 g/L-40 g/L fruit juice, which is one or more of coconut milk, coconut water, and pineapple juice.

In one embodiment of the present disclosure, the rotation speed of the shaking table is 50-200 r/min, and the culture time is 6-72 h during shaking culture, while the culture time is 24-72 h during static culture.

In one embodiment of the present disclosure, the fermentation medium required for inoculation and fermentation includes 10-100 g/L carbon source, 0.5-20 g/L nitrogen source, and 0.5-20 g/L inorganic salt; the initial pH range is 2.5-6.5.

The carbon source and nitrogen source in the fermentation medium are replaced and supplemented by 0.8 g/L-40 g/L fruit juice, which is one or more of coconut milk, coconut water, and pineapple juice.

The fermentation medium can also be composed of 50%-70% fermentation waste liquid, 5-15 h/L carbon source, and 0.5-20 g/L nitrogen source, where the fermentation waste liquid is the residual liquid at the end of fermentation.

In one embodiment of the present disclosure, the carbon source is one or more of sucrose, glucose, fructose, galactose, mannose, glacial acetic acid, ethanol, and glycerol; the nitrogen source is one or more of peptone, yeast extract powder, corn steep liquor, urea, ammonium sulfate, ammonium chloride, and diammonium hydrogen phosphate; and the inorganic salt is one or more of sodium salt, sulfate, phosphate, carbonate, and dihydric phosphate.

In one embodiment of the present disclosure, the container box includes a box body and a lid; the lid is sealed after 1-10 air holes are opened, and the air holes are respectively provided with a microporous filter membrane, a breathable membrane, or a breathable valve, or the lid and the box adopt a labyrinth ventilation mode; and the upper mouth of the box body is provided with a sealing strip, which is closely combined with the lid to form a sealed but breathable environment, so that the liquid is not easy to spill during the moving process after stacking.

The bottom of the box body is provided with supporting feet, which can ensure that the air holes on the lid will not be covered after the box body is stacked.

In one embodiment of the present disclosure, the microporous filter membrane is one of polytetrafluoroethylene (PTFE) membrane, nylon membrane, polyvinylidene fluoride membrane, or mixed cellulose ester; the micropore diameter of the microporous filter membrane is greater than 0.01 mm; and the breathable membrane and the breathable valve have the air permeability of 200-8000 mL/min and are made of one of polyethylene (PE), polypropylene (PP), and polytetrafluoroethylene (PTFE), which have the properties of water resistance and air permeability.

The third purpose of the present disclosure is to provide a Nata fiber prepared by fermentation of a mixed microbial agent with a high yield of bacterial cellulose.

### Beneficial Effects

Beneficial technical effects of the present disclosure are as follows:

The fermentation is carried out in a closed but breathable container box, so that the produced bacterial cellulose is stable and not easily influenced by other miscellaneous bacteria; the closed environment used for fermentation can effectively prevent bacterial cross-contamination; and the container box is specially designed; the upper mouth of the box is provided with a sealing strip, which is closely combined with the lid, so that the sealing performance is good, the liquid is not easy to spill during the moving process, and layer-by-layer accumulation can be realized to greatly facilitate industrial handling and make full use of fermentation room space.

By adopting the method for producing Nata fiber of the present disclosure, the highest yield of bacterial cellulose can reach 20 g/L. By optimizing the components of expansion culture medium and adopting shaking culture, the bacterial concentration of seed culture is greatly improved, so that bacterial cellulose can be produced more efficiently. And the components of fermentation medium are also optimized to further improve the bacterial cellulose yield.

The mixed microbial agent of the present disclosure has good performance and production efficiency, is beneficial to industrial large-scale production, and has obvious economic and social benefits. Nata fiber produced by this microbial agent can be widely used in food, medicine, papermaking and other fields, and has the characteristics of high water holding capacity and high gel strength.

### BRIEF DESCRIPTION OF THE DRAWINGS

FIG. 1 shows the yield of the first batch of primary screening strains in Example 1;

FIG. 2 shows the yield of the second batch of primary screening strains in Example 1;

FIG. 3 shows the yield of the rescreened strain in Example 1;

FIG. 4 shows the scanning electron microscope (SEM) image of the diluted Nata fermented with the mixed microbial agent in Example 5;

FIG. 5 shows the stacking diagram of fermentation containers;

In the figure, 1, lid, 2, air hole, 3, box body, 4, supporting foot.

### DETAILED DESCRIPTION OF THE EMBODIMENTS

In the following, the invention will be described in detail with the drawings and examples.

Komagataeibacter oboediens JGG1 is preserved in the China Center for Type Culture Collection (CCTCC) with the deposit number of CCTCC NO: M 20221774; the preservation date is Nov. 11, 2022, and the preservation address is Wuhan University, Wuhan, China.

Leuconostoc mesenteroides subsp. mesenteroides JGG2 is preserved in CCTCC with the deposit number of CCTCC NO: M 20221775; the preservation date is Nov. 11, 2022, and the preservation address is Wuhan University, Wuhan, China.

A calculation method of bacterial cellulose yield is as follows: a bacterial cellulose membrane is generated after static culture for 6-8 g at constant temperature and floating on the liquid surface; after the membrane is taken out, it is rinsed with water for several times to remove the culture medium and impurities on the surface of the membrane, weighed and recorded as wet weight; the membrane in soaked in 0.1 mol/L NaOH solution and boiled at 100°° C. for 20 min to remove the bacteria and residual culture medium in the liquid membrane, and make the membrane translucent; the membrane is rinsed with distilled water for several times, soaked and neutralized in 0.1 g/L citric acid for 5-10 min after cooling, and rinsed with distilled water for several times until a pH value of 7.2 is measured by lightly pressing the membrane with pH test paper. The membrane is dried at 80° C. for 12 h, cooled to room temperature, weighed, and recorded as dry weight. The yield of cellulose is expressed as g/L (cellulose/medium).

$$Bacterial cellulose yield wet weight ( g / L ) = wet membrane weight / fermentation fluid volume .$$

$$Bacterial cellulose yield dry weight ( g / L ) = dry membrane weight / fermentation fluid volume .$$

The method for detecting the hardness and chewiness of Nata is as follows:

(1) cutting the Nata to be detected into uniform sizes;

(2) selecting a P/0.5, P/25, or P/35 cylindrical probe and selecting a texture profile analysis procedure, where the testing speed is 0.5-10 mm/s, the compression ratio is 10%-90%, the residence time is 1-10 s, and the initial force is 1-30 g;

(3) putting the cut Nata on the test platform and squeezing it twice to get the data of hardness and chewiness.

### Example 1

A method for producing Nata fiber with a mixed microbial agent with a high yield of bacterial cellulose includes the following steps:

(1) Preparation of seed culture: through screening, separation, and purification, oblique strains of Komagataeibacter oboediens and Leuconostoc mesenteroides were obtained, and the two strains were respectively cultured in an expansion culture medium, with the yield increased 10-20 times per generation; the culture temperature was 30° C., and each generation was cultured for 2 d, and the required seed culture was obtained through two generations of subculture, where the bacterial concentration of the seed culture was 10⁷-10⁸ cfu/mL.

The expansion culture medium required for the preparation of seed culture was composed of 80 g/L glucose, 0.1 g/L soy peptone, 2 g/L glacial acetic acid, 2 g/L magnesium sulfate heptahydrate, 20 g/L coconut milk, and water, with an initial pH of 5.0. The culture method was static culture, and the culture time was 72 h.

(2) Inoculation and fermentation: a fermentation medium was prepared and inoculated with the seed culture according to 10% by weight, where the seed culture was prepared by mixing a seed culture of Komagataeibacter oboediens and a seed culture of Leuconostoc mesenteroides at a mass ratio of 1:0.2; after stirring evenly, the fermentation solution was put into a container box, and the lid of the container box was punched with 4 holes; the static culture was conducted at 30° C. for 8 d to obtain a high yield of Nata fiber. The cellulose membrane obtained in this example was translucent and uniform in texture.

The fermentation medium was composed of 30 g/L sucrose, 20 g/L glucose, 0.1 g/L soy peptone, 3 g/L glacial acetic acid, 2 g/L magnesium sulfate heptahydrate, 2 g/L potassium dihydrogen phosphate, 20 g/L coconut milk, and water, with an initial pH of 3.5.

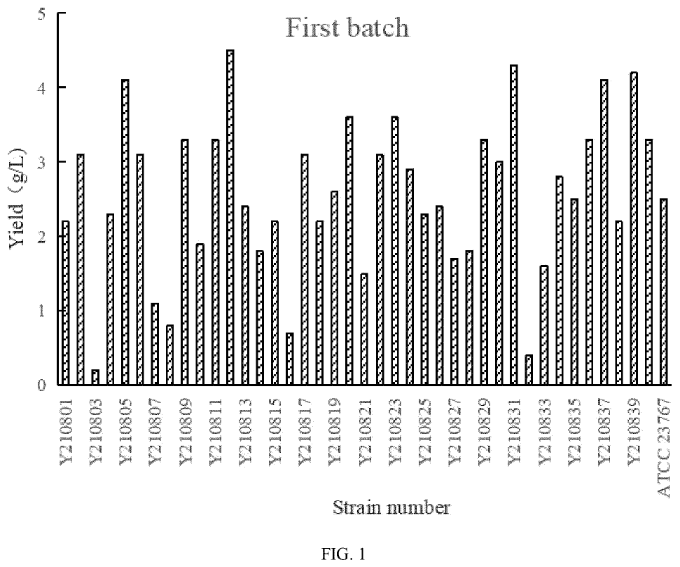

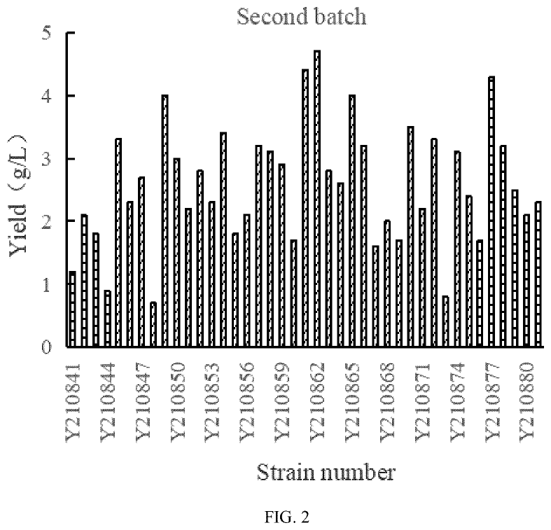

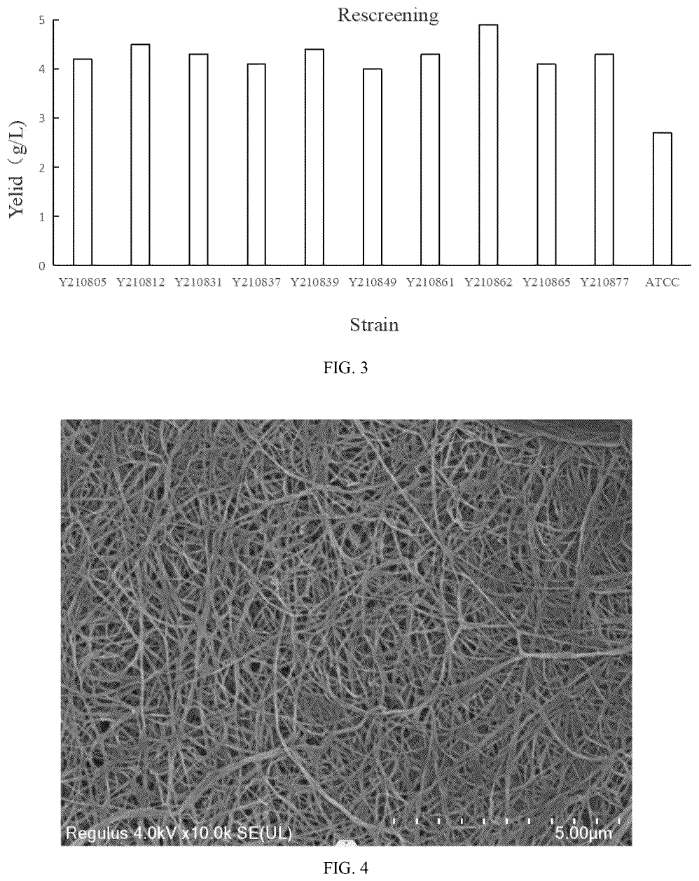

FIG. 1 and FIG. 2 show the yield of the first and second batches of the Komagataeibacter oboediens strains preliminarily screened in Example 1. The dry weight of bacterial cellulose of the strains with higher yield can reach 4.2 g/L-4.8 g/L. FIG. 3 shows the yield of rescreened strains in Example 1. The primary screening strains with higher yield were selected for rescreening, and the yield of rescreened strains was relatively stable at 4 g/L-5 g/L. In this example, the colonial morphology of Komagataeibacter oboediens strains was light yellow, round, raised, and with neat edges.

### Example 2

A method for producing Nata fiber with a mixed microbial agent with a high yield of bacterial cellulose includes the following steps:

(1) Preparation of seed culture: one loop of the oblique strains of Komagataeibacter oboediens and Leuconostoc mesenteroides in Example 1 was selected and inoculated in conical flasks containing 50 mL of expansion culture medium to culture, respectively, with the yield increased 10 times per generation, the culture temperature was 30° C., each generation was cultured for 2 d, and the required seed culture was obtained through two generations of subculture, where the bacterial concentration of the seed culture was 10⁷-10⁸ cfu/mL.

The expansion culture medium required for the preparation of seed culture was composed of 80 g/L glucose, 0.1 g/L soy peptone, 2 g/L glacial acetic acid, 2 g/L magnesium sulfate heptahydrate, 20 g/L coconut milk, and water, with an initial pH of 5.0. The culture method was shaking culture, the rotation speed was 100 r/min, and the culture time was 24 h.

(2) Inoculation and fermentation: a fermentation medium was prepared and inoculated with the seed culture according to 5% by weight, where the seed culture was prepared by mixing a seed culture of Komagataeibacter oboediens and a seed culture of Leuconostoc mesenteroides at a mass ratio of 1:0.1; after stirring, the fermentation solution was put into a closed container box with only air permeability (four holes were drilled, and the corresponding four breathable membranes were pasted; the total air permeability was 7200 mL/min), and the static culture was conducted at 30° C. for 8 d to obtain a high yield of Nata fiber.

The fermentation medium was composed of 30 g/L sucrose, 20 g/L glucose, 0.1 g/L soy peptone, 3 g/L glacial acetic acid, 2 g/L magnesium sulfate heptahydrate, 2 g/L potassium dihydrogen phosphate, 20 g/L coconut milk, and water, with an initial pH of 3.5.

### Example 3

A method for producing Nata fiber with a mixed microbial agent with a high yield of bacterial cellulose includes the following steps:

(1) Preparation of seed culture: one loop of the oblique strains of Komagataeibacter oboediens and Leuconostoc mesenteroides in Example 1 was selected and inoculated in conical flasks containing 50 mL of expansion culture medium to culture, respectively, with the yield increased 10 times per generation, the culture temperature was 30° C., each generation was cultured for 2 d, and the required seed culture was obtained through two generations of subculture, where the bacterial concentration of the seed culture was 10⁷-10⁸ cfu/mL. The expansion culture medium required for the preparation of seed culture was composed of 15 g/L sucrose, 55 g/L glucose, 5 g/L ammonium sulfate, 1.5 g/L glacial acetic acid, 2 g/L magnesium sulfate heptahydrate, 20 g/L coconut milk, and water, with an initial pH of 6.0. The culture method was shaking culture, the rotation speed was 80 r/min, and the culture time was 48 h.

(2) Inoculation and fermentation: a fermentation medium was prepared and inoculated with the seed culture according to 5% by weight, where the seed culture was prepared by mixing a seed culture of Komagataeibacter oboediens and a seed culture of Leuconostoc mesenteroides at a mass ratio of 1:0.2; after stirring, the fermentation solution was put into a closed container box with only air permeability (two holes were drilled, and the corresponding two breathable membranes were pasted; the total air permeability was 2400 mL/min), and the static culture was conducted at 30° C. for 8 d to obtain a high yield of Nata fiber. The fermentation medium was composed of 40 g/L sucrose, 5 g/L ammonium sulfate, 3 g/L glacial acetic acid, 2 g/L magnesium sulfate heptahydrate, 2 g/L potassium dihydrogen phosphate, 20 g/L coconut milk, and water, with an initial pH of 3.5.

### Example 4

A method for producing Nata fiber with a mixed microbial agent with a high yield of bacterial cellulose includes the following steps:

(1) Preparation of seed culture: one loop of the oblique strains of Komagataeibacter oboediens and Leuconostoc mesenteroides in Example 1 was selected and inoculated in conical flasks containing 50 mL of expansion culture medium to culture, respectively, with the yield increased 10 times per generation, the culture temperature was 30° C., each generation was cultured for 2 d, and the required seed culture was obtained through two generations of subculture, where the bacterial concentration of the seed culture was 10⁷-10⁸ cfu/mL. The expansion culture medium required for the preparation of seed culture was composed of 15 g/L sucrose, 55 g/L glucose, 5 g/L ammonium sulfate, 1.5 g/L glacial acetic acid, 2 g/L magnesium sulfate heptahydrate, 20 g/L coconut milk, and water, with an initial pH of 6.0. The culture method was shaking culture, the rotation speed was 100 r/min, and the culture time was 24 h.

(2) Inoculation and fermentation: a fermentation medium was prepared and inoculated with the seed culture according to 5% by weight, where the seed culture was prepared by mixing a seed culture of Komagataeibacter oboediens and a seed culture of Leuconostoc mesenteroides at a mass ratio of 1:0.4; after stirring, the fermentation solution was put into a closed container box with air holes, and the static culture was conducted at 30° C. for 8 d to obtain a high yield of Nata fiber. The fermentation medium was composed of 40 g/L sucrose, 5 g/L ammonium sulfate, 3 g/L glacial acetic acid, 2 g/L magnesium sulfate heptahydrate, 2 g/L potassium dihydrogen phosphate, 20 g/L coconut milk, and water, with an initial pH of 3.5.

### Example 5

A method for producing Nata fiber with a mixed microbial agent with a high yield of bacterial cellulose includes the following steps:

(1) Preparation of seed culture: one loop of the oblique strains of Komagataeibacter oboediens and Leuconostoc mesenteroides in Example 1 was selected and inoculated in conical flasks containing 50 mL of expansion culture medium to culture, respectively, with the yield increased 10 times per generation, the culture temperature was 30° C., each generation was cultured for 2 d, and the required seed culture was obtained through two generations of subculture, where the bacterial concentration of the seed culture was 10⁷-10⁸ cfu/mL. The expansion culture medium required for the preparation of seed culture was composed of 80 g/L sucrose, 1.5 g/L glacial acetic acid, 2 g/L magnesium sulfate heptahydrate, 20 g/L coconut milk, and water, with an initial pH of 6.0. The culture method was shaking culture, the rotation speed was 100 r/min, and the culture time was 24 h.

(2) Inoculation and fermentation: a fermentation medium was prepared and inoculated with the seed culture according to 10% by weight, where the seed culture was prepared by mixing a seed culture of Komagataeibacter oboediens and a seed culture of Leuconostoc mesenteroides at a mass ratio of 1:0.2; after stirring, the fermentation solution was put into a closed container box with air holes (two holes were drilled, and the corresponding two breathable membranes were pasted; the total air permeability was 2400 mL/min), and the static culture was conducted at 30° C. for 8 d to obtain a high yield of Nata fiber.

The fermentation medium was composed of 15 g/L sucrose, 35 g/L glucose, 0.1 g/L soy peptone, 5 g/L glacial acetic acid, 2 g/L magnesium sulfate heptahydrate, 2 g/L potassium dihydrogen phosphate, 20 g/L coconut milk, and water, with an initial pH of 3.0.

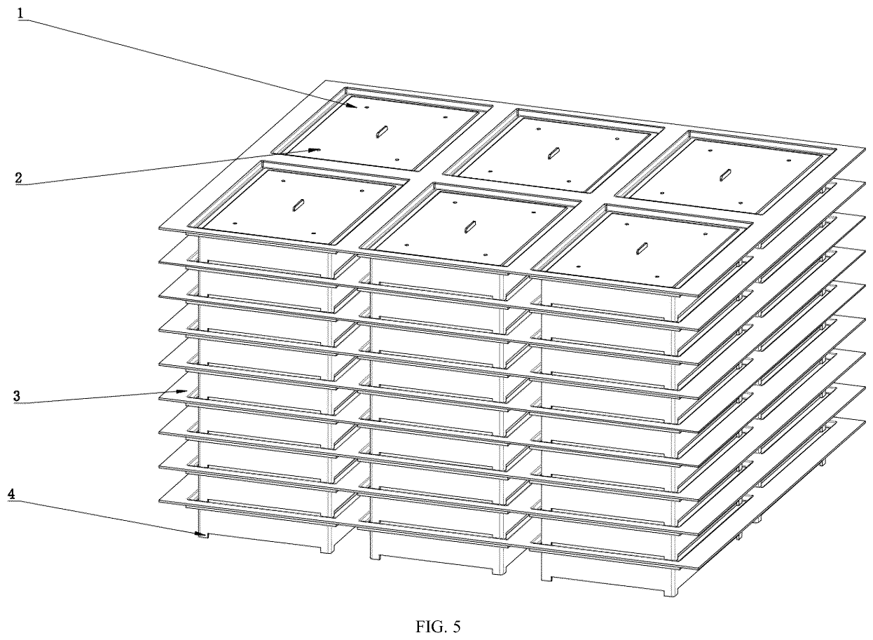

The Nata fiber membrane obtained in this example has a wet membrane thickness of up to 22 mm; FIG. 4 is the SEM image of the sample of Nata fiber obtained in this example. It can be seen from the figure that the fiber filaments produced by static fermentation are tightly interlaced and of uniform thickness, and the diameter of fiber filaments is 20-50 nm, which is smaller than the 50-100 nm commonly mentioned in the literature.

Comparative Example 1

### Production Process of Bacterial Cellulose:

(1) Preparation of seed culture: one loop of the oblique strains of Komagataeibacter oboediens in Example 1 was selected and inoculated in a conical flask containing 50 mL of expansion culture medium to culture, with the yield increased 10 times per generation, the culture temperature was 30° C., and each generation was cultured for 2 d, and the required seed culture was obtained through two generations of subculture, where the bacterial concentration of the seed culture was 10⁷-10⁸ cfu/mL. The expansion culture medium required for the preparation of seed culture was composed of 80 g/L sucrose, 0.1 g/L soy peptone, 2 g/L glacial acetic acid, 2 g/L magnesium sulfate heptahydrate, 20 g/L coconut milk, and water, with an initial pH of 5.0. The culture method was static culture, and the culture time was 72 h.

(2) Inoculation and fermentation: a fermentation medium was prepared and inoculated with the seed culture according to 10% by weight; after stirring, the fermentation solution was put into an open container box, and the static culture was conducted at 30° C. for 8 d to obtain a high yield of bacterial cellulose. The fermentation medium was composed of 50 g/L sucrose, 0.1 g/L soy peptone, 3 g/L glacial acetic acid, 2 g/L magnesium sulfate heptahydrate, 2 g/L potassium dihydrogen phosphate, 20 g/L coconut milk, and water, with an initial pH of 3.5.

### Comparative Example 2

### Production Process of Bacterial Cellulose:

(1) Preparation of seed culture: one loop of the oblique strains of Komagataeibacter oboediens and Leuconostoc mesenteroides in Example 1 was selected and inoculated in conical flasks containing 50 mL of expansion culture medium to culture, respectively, with the yield increased 10 times per generation, the culture temperature was 30° C., each generation was cultured for 2 d, and the required seed culture was obtained through two generations of subculture, where the bacterial concentration of the seed culture was 10⁷-10⁸ cfu/mL. The expansion culture medium required for the preparation of seed culture was composed of 80 g/L sucrose, 0.1 g/L soy peptone, 2 g/L glacial acetic acid, 2 g/L magnesium sulfate heptahydrate, 20 g/L coconut milk, and water, with an initial pH of 5.0. The culture method was static culture, and the culture time was 72 h.

(2) Inoculation and fermentation: a fermentation medium was prepared and inoculated with the seed culture according to 10% by weight, where the seed culture was prepared by mixing a seed culture of Komagataeibacter oboediens and a seed culture of Leuconostoc mesenteroides at a mass ratio of 1:0.2; after stirring, the fermentation solution was put into an open container box, and the static culture was conducted at 30° C. for 8 d to obtain bacterial cellulose. The fermentation medium was composed of 30 g/L sucrose, 20 g/L glucose, 0.1 g/L soy peptone, 3 g/L glacial acetic acid, 2 g/L magnesium sulfate heptahydrate, 2 g/L potassium dihydrogen phosphate, 20 g/L coconut milk, and water, with an initial pH of 3.5.

### Comparative Example 3

### Production Process of Bacterial Cellulose:

(1) Preparation of seed culture: one loop of the oblique strains of Komagataeibacter oboediens in Example 1 was selected and inoculated in a conical flask containing 50 mL of expansion culture medium to culture, with the yield increased 10 times per generation, the culture temperature was 30° C., each generation was cultured for 2 d, and the required seed culture was obtained through two generations of subculture, where the bacterial concentration of the seed culture was 10⁷-10⁸ cfu/mL. The expansion culture medium required for the preparation of seed culture was composed of 15 g/L sucrose, 55 g/L glucose, 5 g/L ammonium sulfate, 1.5 g/L glacial acetic acid, 2 g/L magnesium sulfate heptahydrate, 20 g/L coconut water, and water, with an initial pH of 6.0. The culture method was shaking culture, the rotation speed was 100 r/min, and the culture time was 24 h.

(2) Inoculation and fermentation: a fermentation medium was prepared and inoculated with the seed culture according to 20% by weight; after stirring, the fermentation solution was put into a closed container box with a perforated lid, and the static culture was conducted at 30° C. for 8 d to obtain bacterial cellulose. The fermentation medium was composed of 40 g/L sucrose, 5 g/L ammonium sulfate, 3 g/L glacial acetic acid, 2 g/L magnesium sulfate heptahydrate, 2 g/L potassium dihydrogen phosphate, 20 g/L coconut water, and water, with an initial pH of 3.5.

TABLE 1
Bacterial cellulose yield and thickness of different bacterial cellulose production
processes

| | | | | | | Compar- | Compar- | Compar- |
|---|---|---|---|---|---|---|---|---|
| | Example | Example | Example | Example | Example | ative | ative | ative |
| | 1 | 2 | 3 | 4 | 5 | Example 1 | Example 2 | Example 3 |
| Mass ratio | 1:0.2 | 1:0.1 | 1:0.2 | 1:0.4 | 1:0.2 | 1:0 | 1:0.2 | 1:0 |
| Expansion | pH5.0 | pH5.0 | pH6.0 | pH6.0 | pH6.0 | pH5.0 | pH5.0 | pH6.0 |
| culture | | | | | | | | |
| medium | | | | | | | | |
| Culture | static | 100r/ | 80 r/min, | 100r/ | 100r/ | static | static | 100r/ |
| method of | culture | min, 24 | 48 h | min, 24 | min, 24 | culture | culture | min, |
| seed culture | for 72 h | h | | h | h | for 72 h | for 72 h | 24 h |
| Amount of | 10% | 5% | 5% | 5% | 10% | 10% | 10% | 20% |
| fermentation | | | | | | | | |
| inoculum | | | | | | | | |
| Type of | perforated | perforated | perforated | perforated | perforated | open | open | perforated |
| container box | lid | lid | lid | lid | lid | | | lid |
| | | with | with | | with | | | |
| | | ventilation | ventilation | | ventilation | | | |
| | | device | device | | device | | | |
| Nata fiber | 17 | 16.5 | 18 | 16.7 | 22 | 10 | 11 | 14.0 |
| thickness | | | | | | | | |
| (mm) | | | | | | | | |
| Nata fiber | 15.7 | 15.47 | 16.85 | 15.91 | 20.15 | 10.96 | 11.5 | 13.9 |
| yield dry | | | | | | | | |
| weight (g/L) | | | | | | | | |
| Nata fiber | 760.1 | 747.3 | 791.5 | 772.5 | 886.6 | 521.2 | 552.5 | 610 |
| yield we | | | | | | | | |
| weight (g/L) | | | | | | | | |
| Hardness | 700.5 | 650.8 | 630.2 | 650.7 | 680.3 | 500.9 | 550 | 610.5 |
| (KN/m) | | | | | | | | |
| Chewiness | 218.7 | 197.5 | 210.1 | 207.5 | 226.7 | 135.3 | 162.7 | 174.3 |
| (N) | | | | | | | | |
| Nata fiber gel | 97.93 | 97.93 | 97.87 | 97.94 | 97.73 | 97.90 | 97.92 | 97.72 |
| moisture | | | | | | | | |
| content (%) | | | | | | | | |

$$Moisture content = ( wet weight - dry weight ) / wet weight \times 100 \%$$

As can be seen from Table 1, the wet weight of Nata fiber can be obviously increased by 46%-70%, and the hardness and chewiness can be improved by mixing the bacteria solutions of Komagataeibacter oboediens and Leuconostoc mesenteroides according to the mass ratio of 1:0.1, 1:0.2, and 1:0.4 as the seed culture for fermentation. In addition, the wet weight of Nata fiber can be increased by 22%-45% by optimizing the ventilation mode of the fermentation container, drilling holes in the lid, installing a ventilation device, and controlling the air permeability. In conclusion, the mixture of bacteria solutions of Komagataeibacter oboediens and Leuconostoc mesenteroides in proportion can achieve increased production of Nata fiber. Using a specially designed box for fermentation (as shown in FIG. 5), the wet weight of Nata fiber could reach 886.6 g/L, and the dry weight could reach 20.15 g/L, which was significantly higher than the yield of conventional type strains and had the advantage of high yield.

The above examples are only the better examples given to fully illustrate the invention, and the scope of protection of the invention is not limited to this. The equivalent substitution or transformation made by technicians in the technical field on the basis of the invention is within the scope of protection of the invention. The scope of protection of the invention is subject to the claims.

What is claimed is:

1. A mixed microbial agent with a high yield of bacterial cellulose, comprising Komagataeibacter oboediens and Leuconostoc mesenteroides, wherein a mass ratio of the Komagataeibacter oboediens to the Leuconostoc mesenteroides is 1:(0.1-0.5), and a total viable count is 10⁷-10⁸ cfu/mL.

2. The mixed microbial agent with the high yield of bacterial cellulose according to claim 1, wherein the Komagataeibacter oboediens is named as Komagataeibacter oboediens JGG1, and a deposit number is CCTCC NO: M 20221774.

3. The mixed microbial agent with the high yield of bacterial cellulose according to claim 1, wherein the Leuconostoc mesenteroides is named as Leuconostoc mesenteroides subsp. JGG2, and a deposit number is CCTCC NO: M 20221775.

4. A method for producing Nata fiber using the mixed microbial agent according to claim 1, comprising the following steps:
(1) seed culture preparation: inoculating the Komagataeibacter oboediens and the Leuconostoc mesenteroides into test tubes containing an expansion culture medium, respectively, increasing a yield by 10-20 times per generation, culturing at 25° C.-35° C. for 2-4 d per generation, and obtaining a required seed culture through 2-5 generations of subculture, wherein a bacterial concentration of the required seed culture is 10⁷-10⁸ cfu/mL;
(2) inoculation and fermentation: mixing seed cultures of the Komagataeibacter oboediens and the Leuconostoc mesenteroides according to the mass ratio of 1:(0.1-0.5), preparing a fermentation medium, inoculating a mixed seed culture according to 1%-20% by weight, after stirring, filling a fermentation solution into a container box, and conducting static culture at 25° C.-35° C. for 6-8 g to obtain a high yield of Nata fiber.

5. The method according to claim 4, wherein the expansion culture medium required for the seed culture preparation comprises 10-100 g/L carbon source, 0.5-20 g/L nitrogen source, and 0.5-20 g/L inorganic salt, and an initial pH range is 2.5-6.5; and a culture method is shaking culture or the static culture.

6. The method according to claim 5, wherein the shaking culture is conducted for 6-72 h with a rotation speed of a shaking table of 50-200 r/min; and the static culture is conducted for 24-72 h.

7. The method according to claim 4, wherein the fermentation medium required for the inoculation and fermentation comprises 10-100 g/L carbon source, 0.5-20 g/L nitrogen source, and 0.5-20 g/L inorganic salt, and an initial pH range is 2.5-6.5.

8. The method according to claim 5, wherein the carbon source is one or more of sucrose, glucose, fructose, galactose, mannose, glacial acetic acid, ethanol, and glycerol; the nitrogen source is one or more of peptone, yeast extract powder, corn steep liquor, urea, ammonium sulfate, ammonium chloride, and diammonium hydrogen phosphate; and the inorganic salt is one or more of sodium salt, phosphate, sulfate, carbonate, and dihydric phosphate; and
the carbon source and the nitrogen source are supplemented by 0.8 g/L-40 g/L fruit juice, wherein the fruit juice is one or more of coconut milk, coconut water, and pineapple juice.

9. The method according to claim 4, wherein the container box comprises a box body and a lid, the lid is sealed after 1-10 air holes are opened, and the air holes are respectively provided with a microporous filter membrane, a breathable membrane, or a breathable valve, or the lid and the box body adopt a labyrinth ventilation mode; and an upper mouth of the box body is provided with a sealing strip, and the sealing strip is closely combined with the lid to form a sealed but breathable environment, so that a liquid is not easy to spill during a moving process after stacking.

10. The method according to claim 9, wherein the microporous filter membrane is one of a polytetrafluoroethylene (PTFE) membrane, a nylon membrane, a polyvinylidene fluoride membrane, or mixed cellulose ester; a micropore diameter of the microporous filter membrane is greater than 0.01 mm; and the breathable membrane and the breathable valve have an air permeability of 200-8000 mL/min and are made of one of polyethylene (PE), polypropylene (PP), and PTFE, and the breathable membrane and the breathable valve have properties of water resistance and air permeability.

11. The method according to claim 7, wherein the carbon source is one or more of sucrose, glucose, fructose, galactose, mannose, glacial acetic acid, ethanol, and glycerol; the nitrogen source is one or more of peptone, yeast extract powder, corn steep liquor, urea, ammonium sulfate, ammonium chloride, and diammonium hydrogen phosphate; and the inorganic salt is one or more of sodium salt, phosphate, sulfate, carbonate, and dihydric phosphate; and
the carbon source and the nitrogen source are supplemented by 0.8 g/L-40 g/L fruit juice, wherein the fruit juice is one or more of coconut milk, coconut water, and pineapple juice.
